(19) United States
(12) Patent Application Publication
(10) Pub. No.: US 2011/0195507 A1
(43) Pub. Date: Aug. 11, 2011

(54) METHOD AND APPARATUS FOR DETECTING DATE RAPE DRUGS IN A LIQUID

(76) Inventor: Donna Dancer, Veman, AZ (US)

(21) Appl. No.: 13/083,580

(22) Filed: Apr. 10, 2011

Related U.S. Application Data
(63) Continuation of application No. 12/121,872, filed on May 16, 2008.

(51) Int. Cl. G01N 21/78; G01N 33/52

(52) U.S. Cl. 436/24; 422/419; 422/420; 436/20

(57) ABSTRACT

The present invention is a testing apparatus and method for detecting the presence of a date rape drug such as flunitrazepam or gamma hydroxyl butyrate in a liquid, such as an alcoholic beverage. The testing apparatus includes a base member and a flap member removably attached to the base member. The testing apparatus also includes at least one testing stick enclosed within the base member. The testing sticks include an elongated member and a chemical reagent disposed on the distal end or anywhere along the elongated member. The chemical reagents utilized to detect the presence of flunitrazepam are Zimmerman's Test reagent methanolic 2,4-dinitrobenzene and potassium hydroxide and the chemical reagent for GHB is 5,5 Dibromo-o-cresolsulfonphthalein. The chemical agent reacts with the date rape drug to produce a change in color of the chemical reagent indicating the presence of the date rape drug in the liquid.

This application is a continuation application that claims priority to U.S. application Ser. No. 12/121,872 filed on May 16, 2008, the entire disclosure of which is incorporated by reference.

### FIELD OF THE INVENTION

The present invention relates generally to a method and an apparatus for detecting the presence of date rape drugs in a liquid, such as a beverage.

### BACKGROUND OF THE INVENTION

With an increase in the trend of dating among adolescents and the grown-up population, cases of sexual assaults while dating have also witnessed an increase. Sometimes perpetrators committing such sexual assaults use toxic or harmful substances or date rape drugs to temporarily retard the physical and psychological activity of their victims. Examples of such date rape drugs include flunitrazepam, available under the trade name Rohypnol, and gamma hydroxyl butyrate or GHB. These drugs usually have applications in medical treatments and consequently are available at medical facilities and pharmacies. Rohypnol is a white tablet that is used in medicinal science for treating severe sleep disorder and GHB, also known as liquid ecstasy, is a naturally occurring metabolite and is used in medicinal science for treating neurological disorders, enhancing muscle strength and increasing the rate of release of growth hormones.

However, an inappropriate dose of these drugs is known to prevent generation of sensory impulses or responses by the victims after consumption. For example, Rohypnol has no taste or odor and on consumption without any medical supervision or prescription, the chemical drug may cause harmful effects such as drowsiness, impaired motor skills, impaired judgment, reduced inhibition, dizziness and confusion. Similarly, GHB has the capacity to produce intoxication and amnesia. Furthermore, a combination of the two chemical drugs in a beverage may serve as a partial anaesthetic, thereby causing partial or full amnesia. More often than not, these drugs may be mixed with alcoholic beverages to affect the victims to an even relative greater extent.

Administration of these chemical drugs in beverages results in producing the aforementioned harmful effects on a victim who may gradually be targeted for a sexual assault, or robbery, or any other such incidence. Furthermore, the harmful effects of the chemical drugs may start within 15 minutes to 20 minutes after consumption, thereby providing a short time for the victim to raise a safety alarm. The aforementioned effects become much more severe when the chemical drugs are administered in heavy doses to the victim. Furthermore, administration of a heavy dose of the chemical drugs may result in permanent psychological damage to the victim and in some cases an overdose may result in instant death of the victim.

Accordingly, various testing apparatuses and methods have been employed for detecting the date rape drugs, such as the afore-mentioned chemical drugs in a liquid such as a beverage. Such conventional testing apparatuses employ test strips that may work on a principle of a visual indication when dipped in the liquid having the date rape drugs. However, use of such conventional test strips has not proven to be very reliable. Alternatively, some of the conventional testing apparatuses include a hollow casing, a plurality of beads and a test strip. The hollow casing includes an interior volume and an interior surface. A plurality of beads is disposed within the interior volume of the hollow casing. The test strip is disposed on the interior surface of the casing. For detecting the presence of the date rape drugs in the liquid, the testing apparatus is dipped into the liquid. The beads provide a visual indication to show the presence of the date rape drugs in the liquid. Another such complex conventional testing apparatus exists in the form of an analytic testing cassette having different units of analytic devices required for detecting the date rape drugs. However, all such conventional testing apparatuses involve a complex configuration. Furthermore, these conventional testing apparatuses are bulky, expensive, and not easily portable. Furthermore, most of such conventional testing apparatuses are difficult-to-operate.

It is an object of the present invention is to provide an easy, quick and reliable apparatus for detecting the presence of date rape drugs in a liquid, such as an alcoholic beverage or non-alcoholic beverage.

Another object of the present invention is to provide a method for detecting the presence of date rape drugs in a liquid, such as an alcoholic beverage or non-alcoholic beverage.

Accordingly, there is a need to develop a testing apparatus for an easy and quick detection of toxic or date rape drugs that may be present in a liquid prepared for consumption by a user. Furthermore, the testing apparatus should be compact and portable. Furthermore, there is a need for developing a method for detecting the presence of date rape drugs in a liquid in an easy and a reliable manner.

### SUMMARY OF THE INVENTION

In view of the foregoing disadvantages inherent in the prior art, the general purpose of the present invention is to provide a testing apparatus and a method of use thereof for detecting the presence of date rape drugs in a liquid, such as a beverage, which includes all the advantages of the prior art, and overcomes the drawbacks inherent therein.

In one embodiment of the present invention, a testing apparatus is provided for detecting the presence of a date rape drug in a liquid. The testing apparatus includes a base member having a first folded portion and a second folded portion, and a flap member removably attached to the first folded portion of the base member. Furthermore, the testing apparatus includes at least one testing stick enclosed within the base member. The testing stick includes an elongated member and a chemical reagent disposed on a distal end of the elongated member. The chemical reagent is capable of reacting with the date rape drug. The reaction of the chemical reagent with the date rape drug results in a change in color of the chemical reagent indicating the presence of the date rape drug in the liquid.

In one embodiment of the present invention, a testing stick is provided for detecting the presence of a date rape drug in a liquid. The testing stick includes an elongated member that includes a chemical reagent disposed on a distal end of the elongated member. The chemical reagent is capable of reacting with the date rape drug that results in a change in color of the chemical reagent indicating the presence of the date rape drug in the liquid.

In another embodiment of the present invention, a method is provided for detecting the presence of a date rape drug in a liquid. The method includes providing the liquid and inserting at least one testing stick in the liquid. The testing sticks include an elongated member and a chemical reagent disposed on a distal end of the elongated member. The chemical reagent is capable of reacting with the date rape drug. Furthermore, the method includes observing a change in color of the chemical reagent after inserting the testing stick in the liquid. The change in the color indicates the presence of the date rape drug in the liquid.

These and other embodiments of the present invention, along with the various features of novelty that characterize the invention, are pointed out with particularity in the claims annexed hereto and form a part of this disclosure. For a better understanding of the present invention, its operating advantages, and the specific objects attained by its uses, reference should be made to the accompanying drawing and the descriptive matter in which there are illustrated exemplary embodiments of the present invention.

### BRIEF DESCRIPTION OF THE DRAWING

The advantages and features of the present invention will become better understood with reference to the following detailed description and claims taken in conjunction with the accompanying drawing, in which:

FIG. 1 illustrates a front side perspective view of a testing apparatus for detecting the presence of a date rape drug in a liquid, according to one embodiment of the present invention.

FIG. 2 illustrates a flow chart of a method for detecting the presence of a date rape drug in a liquid, according to one embodiment of the present invention.

### DETAILED DESCRIPTION OF THE INVENTION

The exemplary embodiments described herein detail for illustrative purposes are subject to many variations in structure and design. It should be emphasized, however, that the present invention is not limited to a particular apparatus and a method for detecting date rape drugs in a liquid, as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

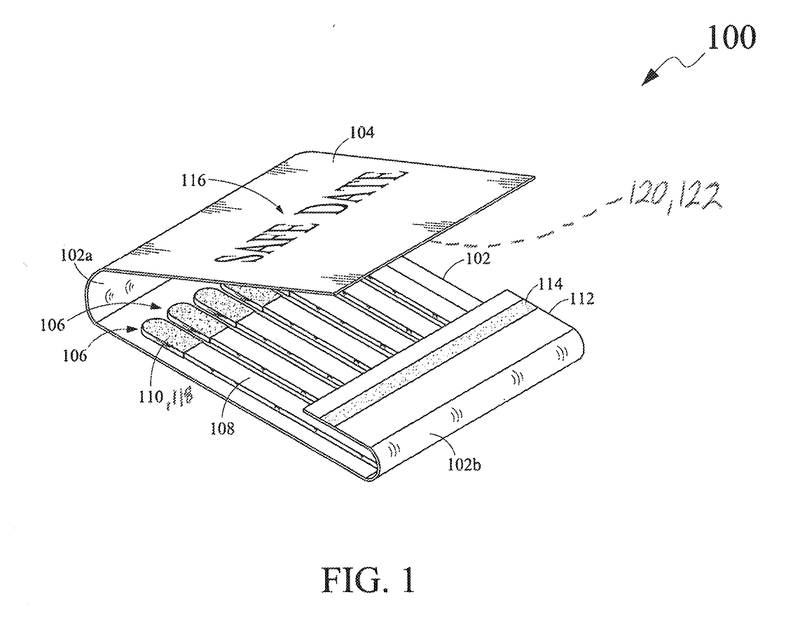

FIG. 1 depicts a perspective view of a testing apparatus 100 for detecting the presence of date rape drugs in a beverage, according to an exemplary embodiment of the present invention. The testing apparatus 100 detects the presence of one or more date rape drugs in a liquid such as an alcoholic beverage, non-alcoholic beverage or other liquid. The term date rape drug refers to toxic substances or chemical drugs that are capable of temporarily retarding physical and psychological activity of a human being. Examples of date rape drugs include, but are not limited to, flunitrazepam, available under the trade name Rohypnol, gamma hydroxyl butyrate or GHB, or a combination of flunitrazepam and GHB. Furthermore, the term liquid refers to liquid substances that are suitable for consumption by humans. Examples of liquid substances may include, but are not limited to, alcoholic beverages and non-alcoholic beverages such as coffee, tea, juices, soft drinks, mixed drinks or beer.

The testing apparatus 100 includes a base member 102 having a first folded portion 102a and a second folded portion 102b. It should be understood that the base member 102 may be made of a structurally strong, lightweight, attractive, and stackable material. More specifically, the base member 102 may be made of a material, such as a cardboard material, a plastic material, or a paper material. For the purpose of this description, the base member 102 is a rectangular cardboard of a dimension that is sufficient enough to hold contents of the testing apparatus 100. The contents of the testing apparatus 100 are explained in detail in the following description of the present invention. It should be well understood that the base member 102, as used herein, may be manufactured in other shapes and sizes.

The testing apparatus 100 includes a flap member 104 removably attached to the base member 102. The flap member 104 is removably attached to the base member 102 through the first folded portion 102a of the base member 102. The flap member 104 provides a covering that protects the contents of the testing apparatus 100. The flap member 104 is a sheet of paper having a rectangular shape and a dimension sufficient enough to cover the contents of the testing apparatus 100. The flap member 104 may be manufactured in other shapes and sizes as well and may be an inherent element of the testing apparatus 100.

The testing apparatus 100 includes at least one testing stick 106 enclosed within the base member 102. The testing sticks 106 are the contents of the testing apparatus 100 and are attached to the base member 102. More specifically, the testing sticks 106 are attached to the base member 102 with an adhesive, which can be any suitable adhesive. Alternatively, the base member 102 may include a plurality of grooves (not shown) for enclosing the testing sticks 106 within the base member 102.

Each of the testing sticks 106 include an elongated member 108 with a distal end 118 and a chemical reagent 110 disposed on the distal end 118. More specifically, the chemical reagent 110 is disposed on the distal end 118. The chemical reagent 110 may also be disposed on the elongated member 108 to completely cover the elongated member 108 or any portion of the elongated member 108 and the distal end 118. Any suitable method may be used for applying the chemical reagent 110 onto the elongated member 108 that include, but are not limited to, coating and impregnation.

The elongated member 108 may be an element that has a relatively larger length than width to provide an easy method for dipping the testing sticks 106 into the beverage so that the chemical reagent 110 is in direct contact with the beverage. Suitable examples of the elongated member 108 include, but are not limited to a stick, a strip or a pipe.

The chemical reagent 110 is capable of reacting with the date rape drugs that are present in a liquid or beverage. The reaction of the chemical reagent 110 with the date rape drugs results in a change in color of the chemical reagent 110 indicating the presence of the date rape drugs in the liquid or beverage. By inserting or dipping the testing sticks 106 in a liquid or beverage to be tested and observing any change in the color of the chemical reagent 110, a person can detect the presence of date rape drugs in the liquid or beverage. The change in the color of the chemical reagent 110 provides a visual indication for the presence of the date rape drugs in the liquid or beverage. The testing stick 106 and chemical reagent 110 may not be toxic to render the liquid or beverage non-drinkable for use after being utilized or added. The chemical reagents 110 include 5,5′ Dibromo-o-cresolsulfonphthalein for gamma hydroxyl butyrate and Zimmerman's Test Reagent methanolic 2,4-dinitrobenzene and potassium hydroxide for Flunitrazepam. After the reaction of the chemical reagent 110 with the date rape drug, a color change may be displayed at the distal end 118 of the testing sticks 106 or where the chemical reagent 110 is otherwise applied.

The testing apparatus 100 may optionally include a support member 112 that helps in holding the testing sticks 106 thereby preventing the testing sticks 106 from dropping-off of the testing apparatus 100. The support member 112 is attached to the base member 102 through the second folded portion 102b of the base member 102 and may include an adhesive layer 114. More specifically, the flap member 104 may be fixed over the support member 112 with the adhesive layer 114 to enclose the testing sticks 106 inside the testing apparatus 100 thereby completely covering the contents of the testing apparatus 100. The flap member 104 is temporarily fixed on the support member 112 such that prior to using the testing sticks 106, the flap member 104 may be easily lifted-up for withdrawing the testing sticks 106 from within the testing apparatus 100. It should also be understood that the testing apparatus 100 may employ an alternate means for temporarily fixing the flap member 104 over the support member 112.

The testing apparatus 100 may include one or more labels, such as a label 116, on an outer surface. More specifically, the label 116 may represent a trade indicia or name imprinted or fixed on the flap member 104. One or more labels 116 may be imprinted or fixed on one or more surfaces of any element of the testing apparatus 100, such as the base member 102, the flap member 104, or the support member 112. It should also be understood that the one or more labels 116 may be designed in standard or custom sizes and shapes, colors, and materials, depending on a manufacturer's preferences.

The testing apparatus 100 may include a plurality of instructions 120 and may illustrate a technique for using the testing apparatus 100 for detecting the presence of the date rape drugs in the beverage. More specifically, the instructions 120 may be imprinted on an inner surface 122 of the testing apparatus 100 during manufacturing. The instructions 120 may include a color scheme depicting an initial color of the chemical reagent 110 before using the testing sticks 106 and a final color of the chemical reagent 110 obtained after using the testing sticks 106.

The testing apparatus 100 serves as a detection system that is shaped as a match book from which a user may withdraw the testing sticks 106 to test ingredients of a beverage. More specifically, the testing sticks 106 also serve as chemically formulated dip sticks enclosed within the matchbook.

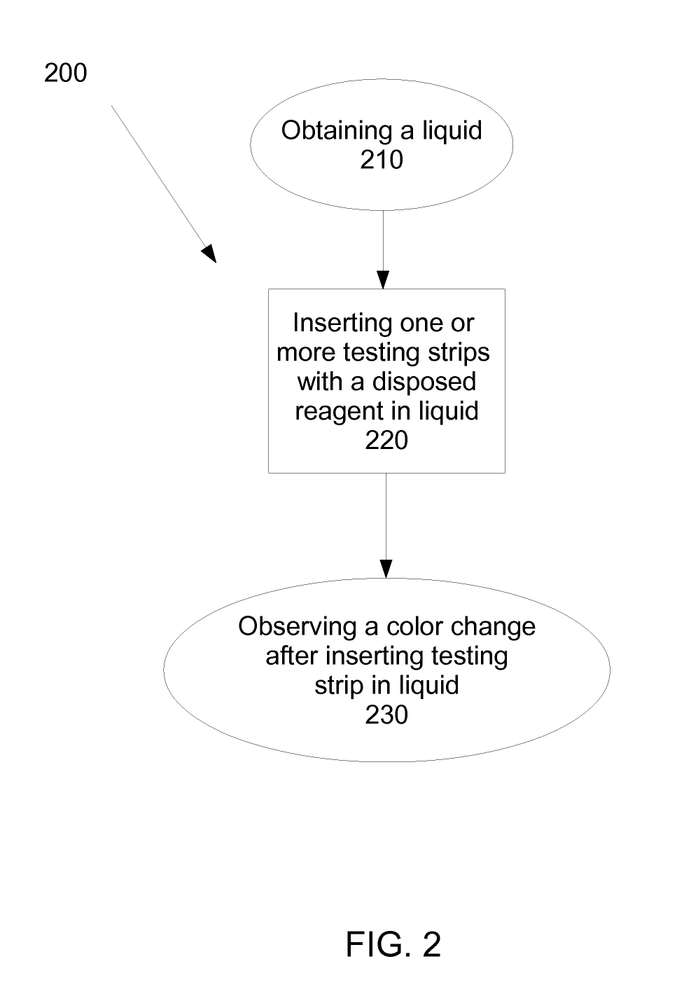

FIG. 2 illustrates a flow chart of a method 200 for detecting the presence of date rape drugs in a liquid, according to one embodiment of the present invention. The method 200 for detecting the presence of date rape drugs in a beverage utilizes the previously described testing apparatus 100 outlined in FIG. 1 and its description. As previously described, the testing apparatus 100 includes at least one testing stick 106 that include an elongated member 108 and a chemical reagent 110 disposed on the distal end 118 of the elongated member 108 or over any portion of the elongated member 108. The chemical reagent 110 is capable of reacting with the date rape drugs that may be present in the beverage. The reaction of the chemical reagent 110 with the date rape drugs results in a change in a color of the chemical reagent 110 and that indicates the presence of the date rape drugs in the beverage. The overall method 200 for detecting the presence of the date rape drugs includes obtaining the beverage to be tested 210, inserting the testing sticks in the beverage 220, observing a possible change in color of the chemical reagent disposed on the testing sticks after inserting the testing sticks in the liquid 230.

The overall method 200 tests for the presence of the date rape drugs flunitrazepam and gamma hydroxyl butyrate. The chemical reagents utilized to detect the presence of flunitrazepam are Zimmerman's Test reagent methanolic 2,4-dinitrobenzene and potassium hydroxide and the chemical reagent for GHB is 5,5′ Dibromo-o-cresolsulfonphthalein.

The change in the color of the chemical reagent indicates the presence of the date rape drugs in the beverage. An indication detecting the presence of the date rape drugs in the beverage may be referred as a positive test. Moreover, no change in the color of the chemical reagent after inserting the testing sticks in the beverage indicates an absence of the date rape drugs in the beverage and such an indication may be referred as a negative test. Accordingly, a positive test of the beverage may indicate to a user that the beverage is contaminated with one of the date rape drugs and is unsafe to consume, whereas a negative test may indicate to the user that the beverage is not contaminated with the date rape drugs and is safe to consume.

The present invention provides a testing apparatus for detecting the presence of date rape drugs, such as flunitrazepam and GHB in a liquid such as a beverage. The testing apparatus includes testing sticks having a chemical reagent that reacts with the date rape drugs that may be present in the beverage. The reaction of the chemical reagent with the date rape drugs results in a change in color of the chemical reagent thereby providing a visual indication for the presence of the date rape drugs in the beverage. The property of the testing apparatus enables a user to ensure safety of the beverage prior to drinking. Therefore, the testing apparatus serves as a detection system that is designed as a match book from which a user may withdraw testing sticks to test ingredients in his or her drink. A change in color of the chemical reagent may alarm the user about the presence of one of the date rape drugs in his or her drink. Moreover, the testing apparatus of the present invention is compact and convenient-to-carry to places such as restaurants, bars and similar places that are available by people to meet for dating purposes.

Furthermore, the testing apparatus is easy and quick-to-use as it does not require following any complex set of instructions prior to use. The present invention also provides a method for detecting the presence of the date rape drugs in the beverage, using the testing apparatus, in a reliable, quick and an easy manner. Therefore, the method and the testing apparatus serve as effective indicating tools for a user for his or her personal safety and security while drinking a liquid in the company of others.

The foregoing descriptions of specific embodiments of the present invention have been presented for purposes of illustration and description. They are not intended to be exhaustive or to limit the invention to the precise forms disclosed and modifications and variations are possible in light of the above teaching. The embodiments were chosen and described in order to explain the principles of the invention and its practical application, and thereby enable others skilled in the art to best utilize the invention and various embodiments with various modifications as are suited to the particular use contemplated.

It is understood that various omissions and substitutions of equivalents are contemplated as circumstances may suggest or render expedient, but such are intended to cover the application or implementation without departing from the spirit or scope of the claims of the present invention.

1. A testing apparatus for detecting the presence of a date rape drug in a liquid, comprising:
a base member having a first folded portion and a second folded portion;
a flap member removably attached to said first folded portion of said base member; and
one or more testing sticks with a distal end enclosed within said base member, said testing sticks include an elongated member and one or more chemical reagents disposed on said distal end, said chemical reagents react with said date rape drug resulting in a change in a color of said chemical reagent indicating the presence of said date rape drug in said liquid.

2. The apparatus according to claim 1, wherein said support member is attached to said base member through said second folded portion with an adhesive layer to enclose said testing sticks.

3. The apparatus according to claim 1, wherein said elongated member is a stick, a strip or a pipe.

4. The apparatus according to claim 1, wherein said date rape drug is flunitrazepamor.

5. The apparatus according to claim 4, wherein said reagent is Zimmerman's Test reagent methanolic 2,4-dinitrobenzene and potassium hydroxide.

6. The apparatus according to claim 1, wherein said date rape drug is gamma hydroxyl butyrate.

7. The apparatus according to claim 5, wherein said reagent is 5,5′ Dibromo-o-cresolsulfonphthalein.

8. The apparatus according to claim 1, wherein one or more labels are disposed on said base member, said support member or said testing sticks.

9. The apparatus according to claim 1, wherein said liquid is selected from the group consisting of alcoholic beverages, non-alcoholic beverages, coffee, tea, juices, soft drinks, mixed drinks or beer.

10. The apparatus according to claim 1, wherein said apparatus is imprinted with a plurality of instructions and an illustration of a technique to utilize said apparatus.

11. A testing stick apparatus for detecting a date rape drug in a liquid, comprising:
an elongated member; and
one or more chemical reagents disposed on said elongated member, said chemical reagent capable of reacting with said date rape drug, wherein said chemical reagent reacts with said date rape drug resulting in a change in color of said chemical reagent indicating said date rape drug being present in said liquid.

12. The apparatus according to claim 11, wherein said elongated member is a stick, a strip or a pipe.

13. The apparatus according to claim 11, wherein said date rape drug is flunitrazepamor.

14. The apparatus according to claim 13, wherein said reagent is Zimmerman's Test reagent methanolic 2,4-dinitrobenzene and potassium hydroxide.

15. The apparatus according to claim 11, wherein said date rape drug is gamma hydroxyl butyrate.

16. The apparatus according to claim 15, wherein said reagent is 5,5′ Dibromo-o-cresolsulfonphthalein.

17. The apparatus according to claim 11, wherein one or more labels are disposed on said base member, said support member or said testing sticks.

18. The apparatus according to claim 11, wherein said liquid is selected from the group consisting of alcoholic beverages, non-alcoholic beverages, coffee, tea, juices, soft drinks, mixed drinks or beer.

19. A method for detecting flunitrazepamor or gamma hydroxyl butyrate in a liquid, comprising:
obtaining said liquid;
inserting one or more testing sticks that includes an elongated member and one or more chemical reagents disposed on said elongated member that include Zimmerman's Test reagent methanolic 2,4-dinitrobenzene and potassium hydroxide that reacts with flunitrazepamor and 5,5′ Dibromo-o-cresolsulfonphthalein that reacts with gamma hydroxyl butyrate resulting in a change in color in said liquid; and
observing said color change after inserting said testing stick in said liquid, indicating whether flunitrazepamor or gamma hydroxyl butyrate is present.

20. The method according to claim 19, wherein said liquid is selected from the group consisting of alcoholic beverages, non-alcoholic beverages, coffee, tea, juices, soft drinks, mixed drinks or beer.
